(19) United States
(12) Patent Application Publication
(10) Pub. No.: US 2011/0190637 A1
(43) Pub. Date: Aug. 4, 2011

(54) MEDICAL MEASURING SYSTEM, METHOD FOR SURGICAL INTERVENTION AS WELL AS USE OF A MEDICAL MEASURING SYSTEM

(75) Inventors: Bruno Knobel, Laufen (CH); Charles Findeisen, Wettingen (CH)

(73) Assignee: Naviswiss AG, Laufen (CH)

(21) Appl. No.: 12/737,812

(22) PCT Filed: Aug. 18, 2009
(86) PCT No.: PCT/EP2009/005979

§ 371 Date: Mar. 31, 2011

(30) Foreign Application Priority Data
Aug. 18, 2008 (DE) 10 2008 038 126.8

(51) Int. Cl. A61B 6/00; H04N 7/18

(52) U.S. Cl. 600/476; 348/77; 348/68; 348/E07.085

(57) ABSTRACT

The invention relates to a medical measuring system, surgical intervention methods, and the use of a medical measuring system. The measuring system comprises two two-dimensional imaging sensors, a light source that is rigidly connected to the camera system in order to project structured light onto objects, and a structure that has a two-dimensional pattern and is mounted on an object.

Tracking systems are used in Computer Assisted Surgery (CAS) in order to determine the spatial position and orientation of instruments or body parts, for instance.

The typical optical tracking systems that are currently available are based on the one hand, on camera systems that are statically positioned at a certain distance from the operation field and on the other hand on locators that are fastened to corresponding body parts or instruments.

In the present application, following the notation of the document WO 2006/131373 A2, the term “tracking system” describes a device by means of which a body is identified optically and a relative movement between it and another body can be tracked.

In the present application, the term “navigation system” describes a device with which the results of the tracking system as well as the spatial information of the body which have been measured by means of other imaging methods such as ultrasound, MRI, or radiography, are available to the surgeon in a common reference system.

In the present application, the term “measuring system” describes a device consisting of a camera system and containing other components such as light sources for a vertical illumination, or means for projection of structured light onto object surfaces and/or video cameras and locators and/or structures with laminar patterns.

The locators usually employed consist of several reflective balls that are rigidly attached to each other or of active markers, mostly consisting of IR light-emitting diodes. The tracking system as well as the locators must have a sufficient geometric size in order to ensure the measuring accuracy of the system. For a sufficient redundancy, the locators must have a correspondingly complex design.

As a rule, the illumination for the locators is integrated into the tracking system. In the prior art, the locators equipped with reflective balls are illuminated with an infrared light. The camera lenses are equipped with corresponding color filters. Practically background-free images of the illuminated reflective balls are thus obtained. Not relevant details of the measurement volume are thus masked which considerably simplifies the analysis of the camera images.

A sufficient infrared illumination of the laminar markers in the measurement volume that do not have reflective surfaces is expensive with the currently available light-emitting diodes. The infrared light can alternately come entirely or partially from the surgical lamp. Modern surgical lamps equipped with light-emitting diodes however do not radiate in the infrared region.

For certain uses, for instance in dental surgery, the typically employed optical tracking systems with corresponding locators are too big and/or too unhandy. A mobile, handy tracking system with a camera system fastened directly on an instrument is described in the international application WO 2006/131373 A2. Adequate structures and patterns are fastened to the objects.

The document WO 2006/131373 A2 does not describe how this camera system can be implemented so that it can be sterilized (for instance in an autoclave) for use in the operating room.

Furthermore, the document WO 2006/131373 A2 does not describe how optical, precise tracking systems that can be sterilized in an autoclave can be implemented in a miniature design.

The dissertation of R. U. Thoranaghatte, University of Bern, 2008, describes a navigation system consisting of an endoscope equipped with a camera and planar patterns attached to the object. The measuring system has practically no redundancy. The measurement accuracy is relatively low: the quality of the measurement results strongly depends on the orientation of the patterns relative to that of the endoscope. Soiled patterns are problematic.

Flat laminar or three-dimensional patterns for navigation systems are known. The publications U.S. Pat. No. 5,792,147, WO 2004002352A2 or U.S. Pat. No. 6,978,167B2 can be mentioned as an example. It is common to all these examples that the high-contrast checkered patterns are reduced to a few marked points, namely the angles of respectively four neighboring squares. This approach requires relatively big surfaces in order to achieve a certain accuracy of the navigation system, which makes relatively big patterns.

The document WO 2006/131373 A2 describes systems and methods which allow the measurement of the surface topology by means of the structured light projected onto the object. These systems are apparatuses that are not connected mechanically to the tracking system. This can be disadvantageous in the field.

Typical optical tracking systems contain no acquisition of the surgical area by means of video cameras integrated into the tracking system.

In surgical orthopedics, implants are used in certain interventions in order to set injured bones in the correct position and orientation. Screws that are placed in predrilled holes in the bone are typically used for setting. For this, the holes must be drilled into the bone optimally with respect to the entry point and the drilling angle. Drilling aids in form of sleeves, which are temporarily fastened to the implants, for instance by means of screwing, are often used for this work step. The optimal areas of the bone for screwing are identified before the intervention by means, for instance, of radiography.

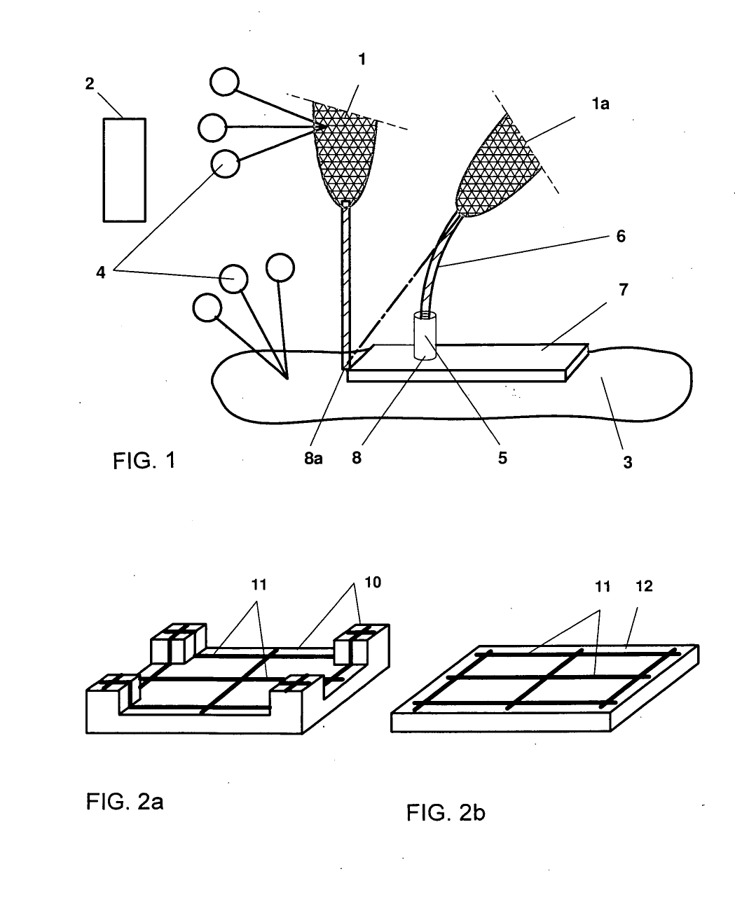

In CAS, the drill 1 is guided in a controlled manner relative to the bone 3 by means of a tracking system 2 (FIG. 1). The drill 1 as well as the bone 3 is thereby equipped with locators 4, so that the tracking system 2 knows the relative orientation and position of the drill 1 with respect to the bone 3 at any time. The position and orientation of the sleeves attached to the implant are usually not measured by the tracking system.

Since the used drills 6 are relatively bendable, the actual contact point 8 defined by the sleeve 5 fastened to the implant, and the drilling direction can deviate from the planned point 8a and direction, if the implant 7 is for instance unintentionally displaced. The position 1a of the drill 1 is measured relative to the bone, which however is not very helpful since the end of the bendable drill is guided through the sleeve 5 typically not measured by the tracking system. A “possible counter-pressing” of the drill based on a visual appraisal by the surgeon also will not help to influence, the position and orientation of the screw hole. Therefore, the possibility that the screws and the implants are not positioned optimally exists.

If drill sleeves are fastened to implants, the axis of the tube guide always lies normally relative to the implant and centered in the position of the corresponding hole for the drill sleeve. The drilling direction is not variable anymore for the subsequent drilling and a non positive-fit of the implant to the bone structure can result in a wrong drilling direction.

Optical camera systems that only identify locators equipped with balls need sensing devices for instance in order to measure the position and orientation of positioned implants. Another method consists in a measurement relative to the locators temporarily attached in a defined manner to the implants.

In surgical orthopedics, synthetic joint sockets are employed for instance, the position and orientation of which can be captured after the setting process by the typical optical tracking systems, for instance with a sensing device.

For different reasons, CAS with optical camera systems is not or hardly used nowadays for surgery of inner organs and soft tissues such as the liver, ligaments of muscles.

In the U.S. Pat. No. 6,424,856B1 a method is described in principle in which CAS can be employed for soft parts.

The technical realization of this method is based on optical tracking systems that were available around the turn of the millennium. More specifically, few discreet marks in form of reflective balls that are attached to the surface of the soft tissues and on the instruments are used.

Since the camera system is positioned statically at a certain distance, these balls have to be relatively big. Small balls are practically not measurable by the camera system.

The accuracy of the system is reduced by soiled ball surfaces.

It is currently not known that this method is widely used in surgery of soft tissues. The available optical tracking systems with the relatively big locators apparently are not very adapted.

Devices and methods with a medical-technical measuring system that do not have or significantly reduce the disadvantages mentioned above are presented in the following.

The medical-technical measuring system has the following components: an optical camera system with at least two laminar imaging sensors, a light source rigidly connected to the camera system for projecting structured light onto objects and a structure with a laminar pattern, that is attached to an object.

A camera system is integrated into the medical-technical measuring system. The camera system consists of at least two, preferably three cameras with laminar sensors and lenses. Many details concerning this are described in the patent application WO 2006/131373 A2. Complementary descriptions of such camera systems are contained in the present application.

The patterns on the structures that are applied onto objects are measured, analyzed and spatially described by the camera system, by means of methods known in photogrammetry.

Following the notation of the document WO 2006/131373 A2, the term “structure” in the sense of the patent application describes a three-dimensional structure, which has a pattern. In the present, a structure in the sense of the patent application is also a body. The structure is for instance designed as a planar plate or as a blunt pyramid.

Following the notation of the document WO 2006/131373 A2, a “pattern” in the sense of the patent application is an image which consists for instance of straight or curved lines of different width and length, of circles, ellipses, triangles and rectangles or of a combination of these. It is preferably applied directly onto a surface of a body or of a structure. It is hereby important that the pattern can be distinguished from a surface on which it is attached by a high contrast.

A most preferable implementation of the medical-technical measuring system is that the structure with a laminar pattern is attached to an object.

A most preferable implementation of the medical-technical measuring system is that the structures are bioresorbable.

A most preferable implementation of the medical-technical measuring system is that the laminar patterns consist of areas of different colors.

The surface consistency of the structures with the applied patterns is very important. Some important aspects of it are described in the following.

The light radiated onto the surface equipped with a highly contrasted pattern is reflected. The reflected light should ideally have Lambert properties.

The light-absorbing pattern parts that can be manufactured in the prior art should ideally not have a radiance that can be measured by the camera system, nor any optical local structures.

Possible visible structures of the surfaces due to the material or the manufacturing process as well as local radiance should not disturb the camera system. Reflective surfaces are more specifically not adapted because of their mechanical and optical properties, if the camera system can resolve details of the surface as well as the glass balls or reflector pyramids.

Following the notation of the document WO 2006/131373 A2, secondary patterns and/or structures in the sense of the patent application, can be identified by other non optical imaging systems such as ultrasound, MRI, CT or C-arm. Secondary patterns and/or structures are disposed in a spatially defined manner relative to the patterns that are measurable by the optical camera system. In a preferred implementation, the high-contrast patterns are applied to the surface of a structure, which itself contains secondary patterns and/or structures.

A most preferable implementation of the medical-technical measuring system is that the structure with a laminar pattern has geometrical and secondary patterns that can be identified by non optical imaging systems, such as an ultrasonic apparatus, MRI, and/or radiographic systems.

Camera systems that are capable of identifying and analyzing laminar surfaces in the measurement volume with many other visible objects, can be employed with an illumination by visible light. Some important aspects for using white light are described in the following.

On the one hand, the white light can come from the surgical lamp itself, for instance. A fill lighting coming for instance from a matrix with light-emitting diodes with a white spectrum integrated into the measuring system can additionally increase the light intensity in the measurement volume. Images with high-contrasted patterns are thus generated. The fill lighting is used for instance during a short exposure time period of the sensors.

A most preferable implementation of the medical-technical measuring system is that it contains lamps for pulsed fill lighting of the object.

The spectrum of the fill light can for instance be the same as that of the surgical lamp. The spectrum of the fill light can alternately be colored, as long as it does not disturb the surgeon.

The intensity and/or pulse duration of the fill light is defined and adjusted dynamically by the camera system itself on the basis of the analysis of the images.

The lenses of the cameras can be equipped with optical band-pass filters. The region of the spectrum for instance that corresponds to a maximal sensitivity of the preferably monochromatic camera sensors can thus be preferred. Another advantage of the optical band-pass filters, is that the color correction of the objectives only needs to be optimal in the selected spectrum of the white light, which allows the use of inexpensive lenses.

The light intensity of the surgical lamp can have a ripple of 100 Hz or 120 Hz for instance, that is caused by its energy supply. It can therefore be advantageous to integrate a light sensor with an evaluation unit into the measuring system, which for instance preferably initiates the synchronization unit of the measuring system for each new image sequence at a maximum light intensity.

The optical band pass filters must let pass the optionally used structured light that is projected onto objects in the measurement volume.

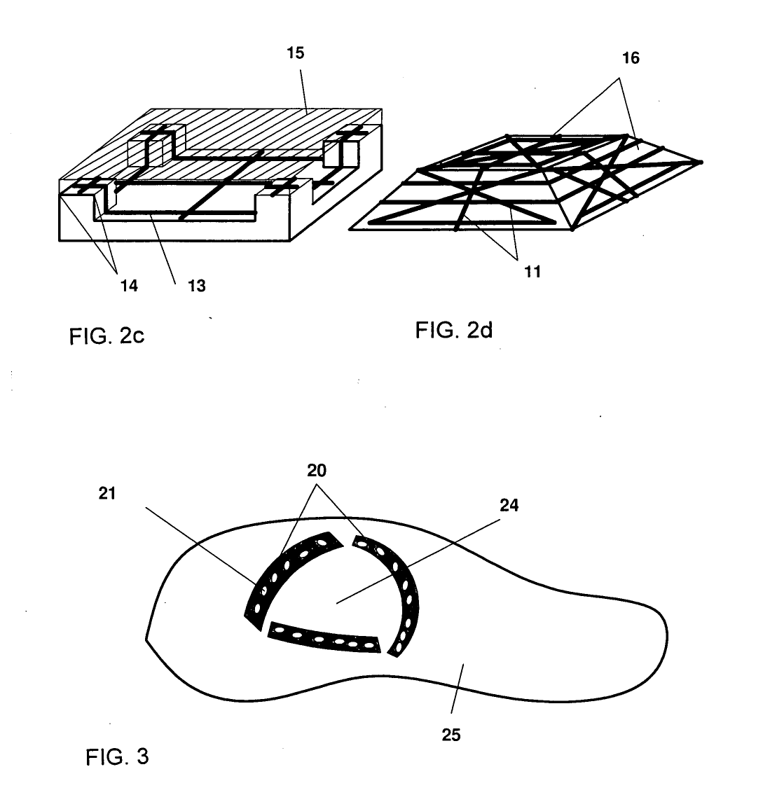

It is known in photogrammetry that the spatial position and orientation of structures with three-dimensionally disposed laminar patterns are in principle identified by the camera system with a higher accuracy than structures with a planar disposed pattern. Two structures with an identical pattern 11, that are disposed on the one hand three-dimensionally 10 on two steps and on the other hand planar 12 on a single level, are represented in the FIGS. 2a and 2b.

Crossed lines with optimal lengths and widths can be advantageous in the design of the patterns, first of all since the intersection can be determined with a higher accuracy by means of adapted algorithms (in comparison with only one circle with a diameter corresponding to the thickness of the lines) and secondly since redundant information is available in order to identify and partially compensate a possible soiling of the patterns. Optionally, the area of the intersection in the pattern can be omitted.

The three-dimensionally disposed patterns can consist of piecewise even patterns 10. They can alternatively be disposed completely on any surface topology adapted to the camera system.

Structures with a three-dimensionally disposed geometrical pattern for instance can be thus implemented with a smaller surface than structures with a planarily disposed pattern, with an otherwise identical accuracy in determining the position and orientation of the patterns.

The three-dimensional structure equipped with patterns can be covered with a transparent protective coating 15, so that the entire pattern is covered and the upper side of the structure is planar and of sufficient optical quality (partially indicated schematically in FIG. 2c). The algorithm of the measuring system takes the optical effect of the transparent protective coating under different viewing angles into account.

This protective coating protects on the one hand the three-dimensional pattern 13 from scratches and soiling and on the other hand prevents for instance the aseptic gloves of the surgeon from being damaged by sharp edges and corners.

Another implementation of a three-dimensional structure is for instance in form of a blunt pyramid of rectangular base (FIG. 2d). Each of the five side surfaces 16 can contain a planar or a three-dimensional pattern. The base of the pyramid thereby lies on the object. A corresponding transparent protective coating can optionally be applied.

A most preferable implementation of the medical-technical measuring system is that the structure with a laminar pattern has geometrical patterns on rigid flat or rigid three-dimensional structures.

Another preferred implementation of patterns on structures is that the prostheses and implants are equipped with patterns in an adequate manner. Based on the patterns analyzed by the camera system, additional mechanical properties of the prostheses and implants are obtained. For instance, the spatial position of the centre of the ball of the implanted synthetic acetabulum relative to a pattern fixed to the pelvis is determined by a measurement.

A most preferable implementation of the medical-technical measuring system is that the laminar patterns are directly attached to implants, prostheses, milling heads and/or rasps.

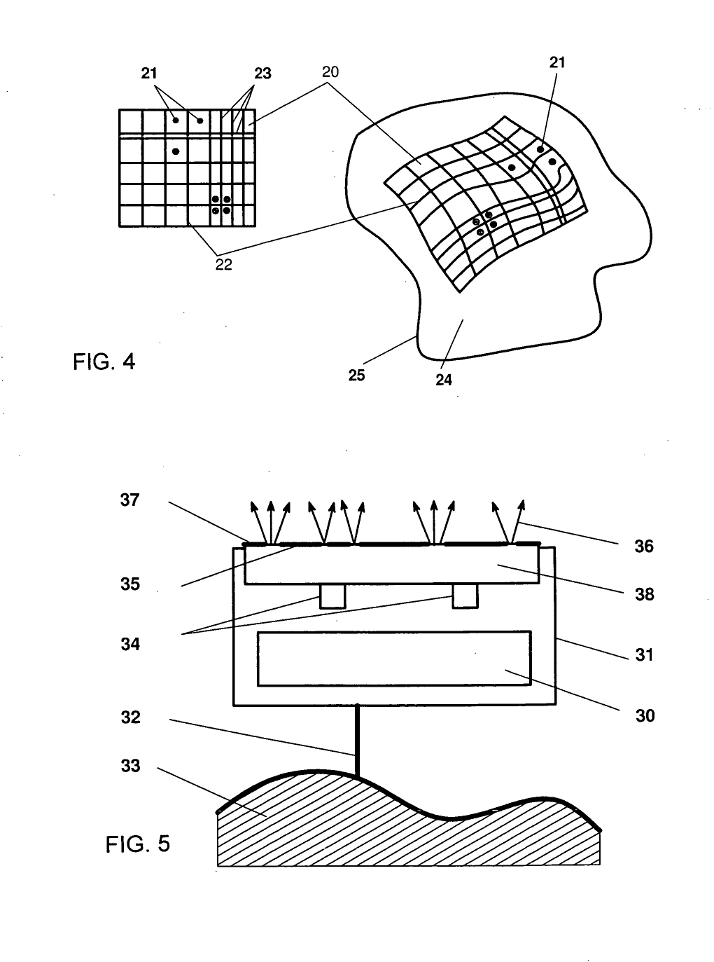

In another preferred implementation, structures with optically high-contrasted geometrical patterns are applied to a flexible and/or expansible foil. Such patterns are exemplarily shown in the FIGS. 3 and 4. These patterns are for instance circular surfaces 21 on a strip with this flexible foil 20, a grid 22 of crossing lines 23 or a dot matrix 21. As exemplarily shown in the FIGS. 2 and 4, these foils are applied to the surface 24 of the preferably deformable objects 25. Distinguished geometrical elements or non equally spaced geometrical elements can serve for instance to orient and identify the pattern or parts of it.

In a preferred implementation, the flexible foil can be configured as a tube that is laid over the corresponding objects.

A most preferable implementation of the medical-technical measuring system is that the patterns are disposed on flexible, deformable structures.

Even pattern areas that are only partly visible to the camera system are sufficient in order to reliably detect the visible relevant surface topology.

The applied pattern is sufficiently redundant. Soiled areas are reliably recognized by the camera system and are correspondingly taken into account in determining the surface topology. A corresponding information can be communicated to the surgeon if necessary, for instance an invitation to clean the foil.

It is advantageous for some uses, if in addition to the optically high-contrasted, laminar forms, the flexible foil contains secondary areas, that are for instance opaque to X-rays, the position of which relative to the geometrical forms is known. By means of radiography, for instance, the secondary areas in the foil can thus be recorded in the liver with respect to the exposed areas in the liver. The optically high-contrasted, laminar forms are herewith also recorded.

The foil can have areas that can be measured by means of ultrasound technology. The position of these areas is known with respect to the geometrical patterns.

A most preferable implementation method for a surgical intervention on a body part with the medical-technical measuring system is that the body surface measured by means of structured light is registered with respect to the data recorded by the other imaging methods such as MRI, ultrasound or radiography.

In another realization of the device, a single foil with many geometrical forms can be replaced by a plurality of separate foils with simple geometrical forms such as circles, for instance. These separate foils 20 are attached individually to the surface of the exposed liver as represented schematically in FIG. 3.

Fixing the flexible structures such as foils, for instance, on the object occurs for instance by means of an adequate biocompatible adhesive.

The applied pattern can be for instance a combination of rigid areas for determining the position and orientation as well as identification and flexible areas with simple patterns for determining the contour of the object.

The mobile measuring system can additionally be equipped with rigidly mounted surgical instruments. The camera system orients itself with respect to the structures attached to the organ. The instrument can be for instance a laparoscopic instrument. It can also be another measuring system such as an ultrasound measuring head, a sensing device, a force measuring system.

Active, i.e. self-luminous, laminar structures emit light signals that are received and analyzed by the camera system. The signals typically lie in the white or infrared region of the spectrum. The energy supply is integrated to the structure's body (also called “marker” in the following) for practical reasons. As represented in FIG. 5, it consists for instance of coin cells 30 usual in commerce that are inserted into the structure's body 31 as required. The structure's body is rigidly fastened to the object 33 by means of an adapted fastener 32.

The light of the active structures is generated for instance by light-emitting diodes (LED) 34 or a light-emitting diode matrix. The geometrical pattern 35 integrated into the structure's body is thus illuminated. The light 36 only exits in the geometrically defined places.

The areas 37 covered by the pattern also absorb the external light that comes from the surgical lamp for instance and impacts on the structure. The contrast of the reproduced pattern thus remains sufficient for the camera system.

A homogenizer 38 thereby generates a constant light density of the light let through by the pattern. A homogenizer is an optical element for instance with a hologram structure or a DOE (diffractive optical element).

Instead of the LEDs and the homogenizer a luminous surface that is realized for instance by means of OLEDs (organic light-emitting diodes) with the pattern, can be employed. The preferred emission spectrum thereby lies in the white, in the colored (green, yellow or red) or in the near infrared region.

The active structures are designed for practical employment in surgery. More specifically, they must be sterilizable. In a preferred implementation, batteries and active electronic elements such as LEDs and power supply are inserted by means of adequate equipment into the sterile casing of the active structure just before use. In another preferred implementation, batteries, LEDs, etc. are employed for high temperatures. The sterilization of the entire active structure is thus possible.

The active structures can be provided for a one-time use, which simplifies the logistics chain. The structures can also be conceived for repeated use.

The active structure according to FIG. 5 generates a light pattern with a constant luminous flux. However, the camera system only uses the light collected by its lenses during the sensor's integration time. The battery of the active structure is thus unnecessarily strained, which reduces the period of use of the active structure. The light flow of the active structure must be sufficient in the entire measurement volume and for any orientation. This presupposes that too much light must thus be generated by the active structure. This also strains the battery.

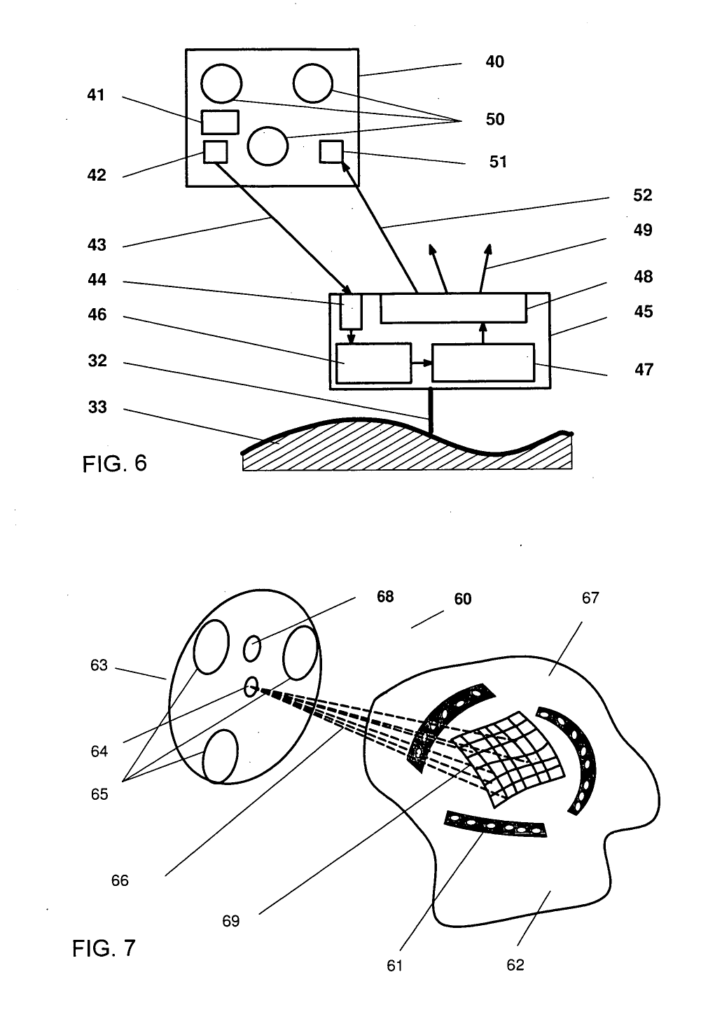

FIG. 6 exemplarily shows how the light generation of the active structure is controlled and synchronized by the measuring system 40. The synchronization module 41 of the measuring system generates the integration time of the camera sensors and the pulse sequences for the signal LED 42. The light signal 43 reaches the light receiver 44 of the active structure 45. A local analysis module 46 analyzes the light signals 43 and generates the corresponding signal in the power module 47 for the lamp 48 with the integrated pattern. The lamp generates the luminous flux 49 that is partially collected by the lenses 50 of the camera system 40 and analyzed. The optional light receiver 51 of the measuring system supports the synchronization module 41 with additional information for the visual signal 52.

The luminous flux pulse of the active structure by no means has to be constant with respect to its intensity and period. The luminous flux of the active structure can change as well in its intensity as in its period for optimal camera images. These values thus typically depend on the orientation and on the place of the active structure inside the measurement volume.

The variations of the intensity as well as of the pulse period do not change much for a defined structure during a series of measuring sequences. The conditions thus do not have to be transmitted to the active structure for each new shot by the camera system. It is thus sufficient for instance to newly transmit the lighting conditions for an active structure after ten shots for instance. Several active structures can be employed simultaneously with a dynamically adjustable luminous flux in the measurement volume.

The chronology of the corresponding control signal of the signal LED of a pulse is for instance an unequivocal signal pattern for the start of a new measurement, followed by pulse sequences for the identification number of an active structure and the required intensity and period of the light emitted by the active structure.

The light collector with the analysis unit (not shown in FIG. 6) determines during the measurement with the measuring system the typical light intensity of the measurement volume. Additionally, it is possible to sequentially read the status of the individual active structure in a diagnosis mode. The light of the active structure is thereby correspondingly modulated, received by the light collector of the measuring system and analyzed by its analysis unit.

These actives structures are optionally equipped with a standby switch, so that they can be activated only when required. The energy supply is thus correspondingly preserved. The logistical handling is also substantially simplified.

The constructive assembly of the active structure allows imaging systems working with X radiation to determine the spatial position and place as well as the identification of the active structure. During constructive assembly, it must be taken into account, that the X-ray opaque (battery, printed circuits, fastening screws) and the X-ray transparent (plastic) materials are correspondingly spatially disposed. This disposition is in a solid geometrical relation to the pattern (for instance the pattern in FIG. 5).

The active structures can be equipped as well with planar patterns as with three-dimensional patterns such as a pyramid for instance.

The patterns of the active structures can for instance be configured by means of control signals, when required. Active structures with OLED patterns for instance are adapted for this. In a preferred implementation the active structures are entirely or partially flexible.

A most preferable implementation of the medical-technical measuring system is that actively radiating structures are provided.

A most preferable implementation of the medical-technical measuring system is that the active structures have an autonomous energy supply.

The device of the measuring system 60 consists, as exemplarily shown in FIG. 7 with laminar patterns on flexible foils 61 on deformable objects 62, of an optical, mobile camera system 63 with the lenses 65, which measures the spatial orientation and position of laminar patterns, and a light source 64 integrated into the measuring system that projects structured light 66 onto the surface 62 of the object 67 equipped with laminar structures 61. The direction of the emitted structure light 66 is defined with respect to the camera system 63. In another preferred implementation this light source is a laser, the beam of which is structured for instance by means of a matrix of micro-lenses. In another preferred implementation, this light source is a laser, the beam of which is structured for instance by means of a DOE (diffractive optical element). In another preferred implementation, the DOE is replaced by a scanner with at least one defined tiltable mirror. Time-varying light structures can be projected therewith onto the object 67. In another preferred implementation, the DOE is replaced by a dynamically configurable mirror matrix. Varying light structures can be projected therewith onto the object 67. In another preferred implementation, several different-colored light sources with a structured light are built into the camera system that is equipped with color sensors. The possibility to respectively use the optimal light source for the measurement of the surface topology of an object thus exists.

A most preferable implementation of the medical-technical measuring system is, that it has at least one other light source for projecting structured light onto objects.

A most preferable implementation of the medical-technical measuring system is, that the other light source has a different color than that of the first one.

A most preferable implementation of the medical-technical measuring system is, that the other light source projects a different structure than the first one.

A most preferable implementation of the medical-technical measuring system is, that for the projection of structured light onto objects, a beam is projected onto controlled mobile mirrors rigidly connected to the camera system.

A most preferable implementation of the medical-technical measuring system is, that for the projection of structured light onto objects, laser light is projected onto diffractive optical elements (DOE).

The laser beam is directed onto the object surface equipped with structures inside the measurement area of the measuring system. Several light beams 66 of the structured light are exemplarily represented in FIG. 7. A generally three-dimensional light pattern 69 is generated therewith on the surface of the object.

The structure light scattered on the surface is measured by the camera system, analyzed and put in relation with the patterns on the neighboring structures on the object.

By modifying the spatial position and orientation of the measuring system, the pattern with the structured light is moved relative to the structures along the surface. The surface topology inside the desired area is determined therewith relative to the neighboring structures. Chronological variations of the surface topology are measurable with this method.

A most preferable method for a surgical intervention on a body part with the medical-technical measuring system is, that the surface of a body part to be treated is measured locally by means of structured light and is put in spatial relation to an attached structure with a pattern.

A most preferable method for a surgical intervention on a body part with the medical-technical measuring system is, that a part of or the entire surface of articular cartilage is measured locally in three dimensions by means of structured light and the place and size of worn-out articulation parts is localized therewith.

A most preferable method for a surgical intervention on a body part with the medical-technical measuring system is, that the rigid structures or flexible foils with geometrical patterns are anatomical structures and anatomically given patterns.

One or more video cameras 68 are optionally integrated to the measuring system as schematically shown in FIG. 7. The functions of the video camera are image acquisition of the relevant operation area, for instance for a clear representation for navigation purposes on a display integrated into the measuring system, for identifying the object texture of the surfaces, object structures such as blood vessels and/or form and position of implants, as a warning device for correlation analyses of successive images and/or for documentation purposes.

In another preferred implementation of the device, rigid patterns on rigid structures are located on rigid objects.

In another preferred implementation of the device, a display such as a monitor with control elements is integrated into the medical-technical measuring system.

A most preferable implementation of the medical-technical measuring system is, that it has at least one display with control elements rigidly connected to the camera system.

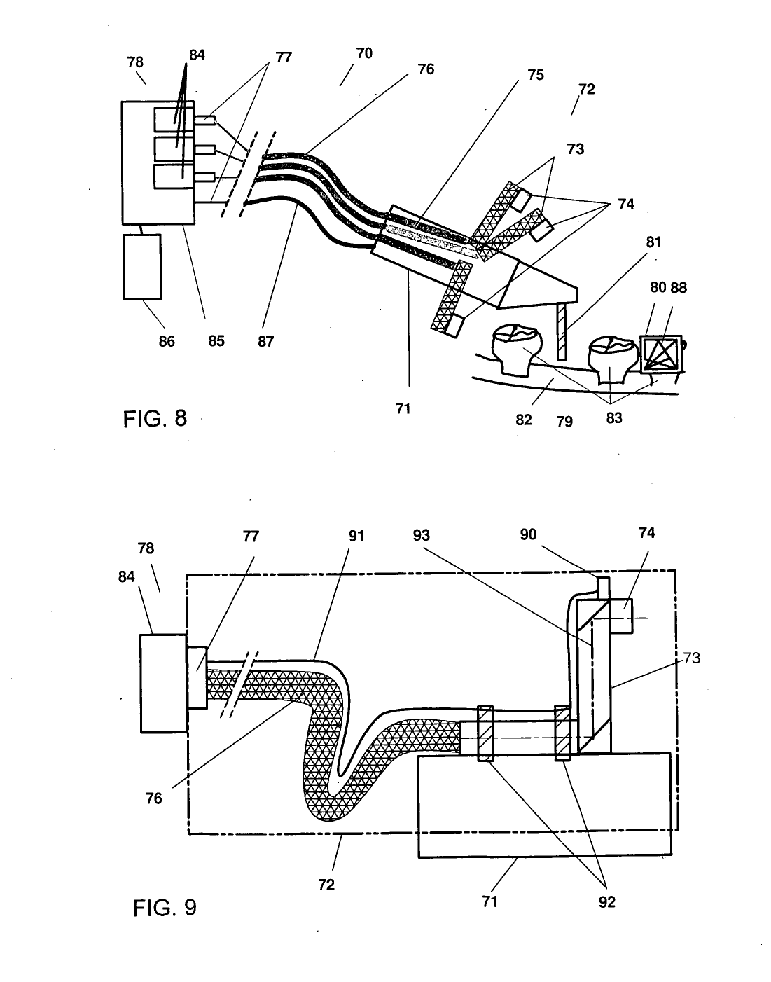

The device schematically shown in FIG. 8 shows a camera system 70 that consists of two parts 72 and 78. The presented example concerns the field of dental surgery with a drilling apparatus 71 as a tool; in principle it can also be deployed in surgical orthopedics or in other surgical fields.

The first, sterile part 72 comprises several periscopes 73 with lenses and lighting device 74, image conductor 75 along the tool 71, a flexible and sufficiently long image guide 76 and an image conductor adapter 77 to the second non sterile part 78 with the sensors 84, the analysis unit 85 and host 86. The number of the periscopes is at least two. In the following a variant with three periscopes is described, since it implies distinct advantages with respect to precision and redundancy and thus security, as opposed to conventional tracking systems. Other connecting cables 87 supply the first part 72 with power, water and compressed air.

The lenses of the periscopes look at the operation field 79. They must more specifically see the structures 80 with the geometrical pattern 88 during the intervention, so that the spatial orientation and position of the tool 71 with the drill 81, the drilling point 82 and the teeth 83 can be determined.

The image conductor 75 is preferably configured with an endoscopic design. The object of the image conductor is to transmit the operation field 79 seen by the lenses on the periscope to the corresponding sensor 84.

As shown schematically in FIG. 9, the lighting device can consist of LEDs 90 with white light, that are attached in an appropriate manner to the periscopes 73 with the optical axes 93 and are supplied with power by means of cables 91. The illumination can also occur directly via the light guide and lenses, the LEDs being located in the second part of the measuring system.

The periscopes are connected mechanically in a defined manner to the tool, for instance with a clip mechanism 92. Several defined fixing positions can be provided for this, so that the lenses of the periscopes are optimally oriented onto the field of work.

This first part of the camera system can be assembled modularly, which makes several options possible.

In a preferred implementation, the lenses of each periscope can be replaceable, so that the camera system has an optimal orientation toward the operation field with respect to distance and visual field. Another option is that the periscopes including the lenses are replaceable. Another option is a replaceable basis of the periscopes, so that the lenses have a sufficient geometrical distance relative to each other. Indeed, with a bigger field of work, the lenses are as a rule correspondingly disposed further apart. The specified precision of the camera system is thus always ensured.

This first part 72 is configured in a sterilizable design. Usually, sterilization is performed by means of autoclaves and also by means of gaseous sterilization. Thereby, it is important that the first part of the camera system is sufficiently sterile for use in the operation field.

A most preferable implementation of the medical-technical measuring system is that respectively one periscope is disposed on the camera system between the sensor and the lenses.

The second part 78 comprises a sensor 84 for each periscope unit, an analysis unit and a visualization device not represented in FIG. 9. This second part does not have to be autoclavable, since it can be positioned sufficiently far from the operation field. The casing of this part can however be externally sterilized with corresponding chemicals, for instance ethylene oxide.

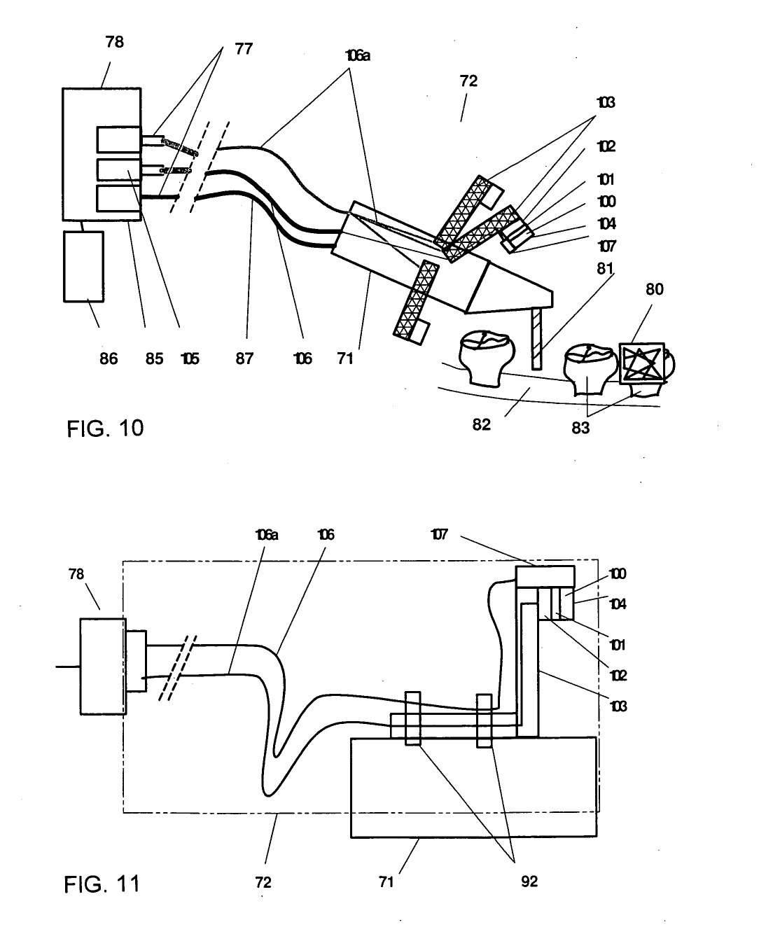

Alternately, lenses 100, optical high-pass filters 104, sensors 101, and a part of the electronic system 102 can be disposed on a mount 103 in the very front of the sterile part 72, as exemplarily represented schematically in the FIGS. 10 and 11. The image conductor is dispensed with. In this device, the light for the illumination is generated in the non sterile part 78 by the lighting module 105 and led to the lighting lenses 107 via light guides 106. In this example, the locally processed sensor information is sent via cables 106a to the electronic system in the non sterile part 78. The components of the first part 72 must be sterilizable in the autoclave.

A most preferable implementation of the medical-technical measuring system is that the camera system has sensors that are spaced or disposed in a star shape, in front of which respectively one lens is disposed.

The cameras can be optionally equipped with optical high-pass filters 104, so that only part of the light spectrum reaches the sensors. This simplifies the layout of the lenses.

Instead of the three individual cameras, a common camera with a correspondingly big imaging sensor can also be used. The three image conductors and periscopes are therewith oriented toward the common imaging sensor of the single camera. The sensor image in this case contains a representation of all three periscopes. This simplifies the constructional expense of the camera system.

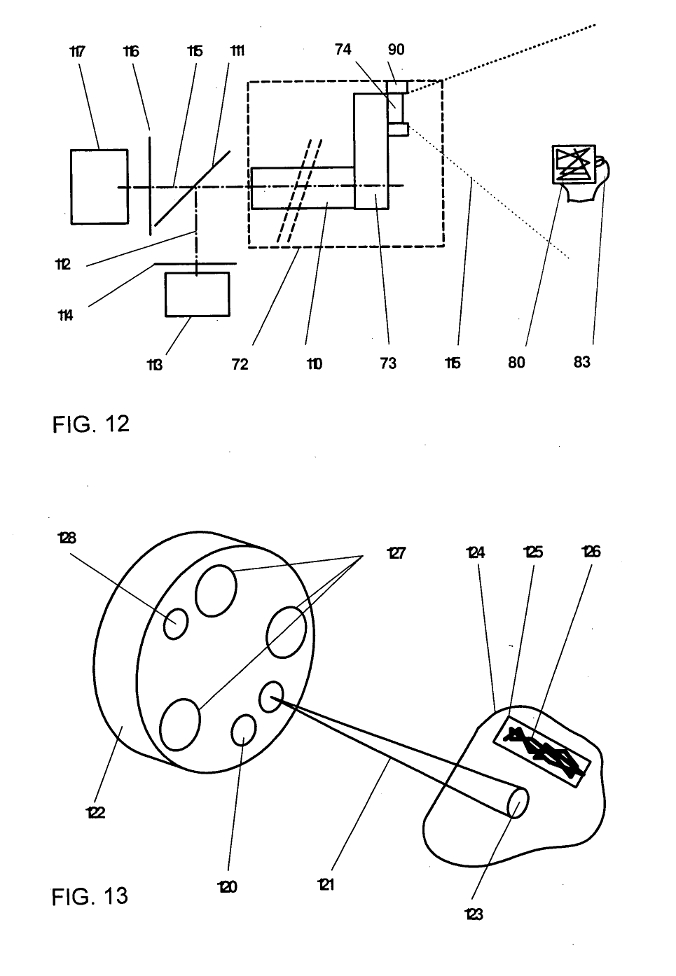

A video camera can be integrated optionally into the device, as represented schematically and exemplarily in FIG. 12, so that the measurement area 115 can be seen via a periscope lens 74. A color video camera is preferably used. In a possible implementation of the device, a beam splitter 111 is integrated at the exit of the image conductor 110 outside the sterile part 72. A part 112 of the light is therewith deflected toward the video camera 113. The optional low pass filter 114 protects the color sensor from infrared light. The other part 115 of the light is let through via the optional color filter 116 to the camera sensor 117.

Another variant is the use of at least two video cameras, which allows a stereo view of the operation field.

It is also possible to integrate video cameras by using a distinct periscope.

A most preferable implementation of the medical-technical measuring system is that it has at least one video camera rigidly connected to the camera system.

Another sensor 120 with its own lens and with an optionally adapted illumination 121 can be integrated into the measuring system as represented schematically in FIG. 13. The object of this sensor is to measure the energy flow as well as for instance the visible light or heat from a certain area 123 of the object. The measurements are brought in local relation to the structure 125 and pattern 126 attached to the object 124 by means of the camera system 127 with an optional illumination 128.

These measurements with the optional sensor are preferably adapted to contactless diagnosis purposes. The optional illumination 121 can be continuous or pulsed. The emitted spectrum can lie in the ultraviolet, visible, near, mid and far infrared and/or terahertz region.

A most preferable implementation of the medical-technical measuring system is that it has a contactless radiation measuring device for the ultraviolet, visible, near, mid and far infrared and/or terahertz spectrum region, rigidly connected to the camera system.

The spectrum measured by the sensor 120 lies in the ultraviolet, in the visible, in the near, mid and far infrared field.

Another preferred spectrum field scanned by the sensor is the terahertz field.

The lens and the sensor attached in front of the sensor must be adapted to the scanned area.

The energy received by the sensor can come from the object itself. In a preferred use, the object can be prepared correspondingly with adequate means for the applied diagnosis. For instance, possible tumor areas can be enriched with specific biological marker substances, and are thus recognized by the sensor 120 under an adequate illumination. In another preferred use, the area to be scanned, for instance a tumor area, is prepared with adequate means and is heated locally by means of irradiated electro-magnetic waves. The heated area is measured by the sensor 120 and put in relation with the available patterns 126 by the measuring system 122. It can also be the energy reflected by the object illuminated by the radiation source.

A preferred implementation of the sensor measures the lateral extension of the scanned area of the surface of the object.

Another preferred implementation measures the temporal behavior of the signal measured by the sensor 120.

Another preferred implementation measures the temporal behavior of the signal measured by the sensor with respect to the irradiated illumination 121.

Another preferred version is a combination of the lateral and the temporal measurement of the energy emitted by the surface of the object.

A most preferable implementation of the medical-technical measuring system is that it has a contactless radiation measuring device for the ultraviolet, visible, near, mid and far infrared and/or terahertz spectrum field, rigidly connected to the camera system.

A most preferable implementation of the medical-technical measuring system is that the radiation measuring device has at least one time and/or spatial resolving sensor.

A most preferable implementation of the medical-technical measuring system is that it has at least one contactless radiation device for the ultraviolet, visible, near, mid and far infrared and/or terahertz spectrum field, rigidly connected to the camera system.

A most preferable method for a surgical intervention on a body part with the medical-technical system is that laterally and/or temporally resolved contactless diagnosis information is put in relation with the spatial topology of the object equipped with the patterns.

As represented schematically in FIG. 4, in another preferred version of the implementation, the energy 129 is radiated behind the object 124 with the freely movable, hand-guided emitter 130. The sensor 120 integrated to the camera system measures the energy emitted by the area 123, that derives from the energy 129 irradiated into it and was modified along the trajectory 131 in the volume of the object.

A source integrated into the camera system alternately emits energy in direction of the object. The intensity of the beam can be laterally and temporally modulated. A sensor positioned behind the object receives the radiation modified by the object.

In a preferred implementation, the entire or a part of the information from the measuring system and the optional sensor is represented in a superimposed manner on the monitor with control elements.

A preferred implementation is a combination of a measuring system with an emitter and a sensor for contactless diagnosis purposes, that are mechanically rigidly connected to medical treatment instruments for lithotripsy or RF-ablation for instance in soft parts, and with patterns attached to the surface of the object.

Another preferred implementation is a combination of a measuring system with an integrated RFID scanner. It is thus possible to communicate with RFID transponders attached to instruments, tools or implants.

A most preferable implementation of the medical-technical measuring system is that it has a RFID scanner rigidly connected to the camera system.

Another preferred implementation of the mobile measuring system is that no connection cables are used for communication and power. All the relevant data for integrated navigation is either loaded beforehand via removable cables, wirelessly and/or with memory cards such as for instance a USB stick onto the camera system or the measured data and/or video sequences are transmitted to the host computer.

Another preferred implementation of a mobile measuring system without a cable for communication and power is that the devices for the fill lighting are not activated or not installed. The local energy supply is thus relieved in favor of a longer operation period of the camera system. A possible consequence of this is that the exposure time of the camera system is longer. Thus, there is a possibility that blurred images are obtained because of relative movements between the measuring system and the object. Blurs caused by the movements of the camera system during the exposure time can be compensated by the measurement and by taking linear accelerations and angular accelerations of the measuring system into account, for instance with MEMS (MEMS=micro-electro-mechanical systems).

A most preferable implementation of the medical-technical measuring system is that an a) an energy source, b) a data storage unit, c) a processor, d) a display and control elements, as well as e) a wireless data communications equipment is disposed as a compact unit with the camera system.

A device in which the position and orientation of the individual drill sleeves relative to the reference pattern are registered by the camera system is schematically presented in the following in FIG. 15.

The device is based on an optical camera system 140 and patterns 141, 142, 143 in place of the locators. The camera system 140 is thereby capable of measuring the position and orientation of the patterns. A structure with a pattern 142 is rigidly disposed on each of the sleeves 144 that are attached to the implant 145. The pattern can optionally be integrated to the surface of the sleeve.

The implant 145 itself can also have such patterns 143 either integrated to the surface or in the form of a removable structure.

Patterns 141 are temporarily attached to the bone surface 146. The orientations and positions of the sleeves, of the implants and of the part of the bone are therewith permanently measured by the measuring system 140 that is fixed to the drilling apparatus 147 with the tool axis 147a. With this device, the surgeon can make precise drill holes 148 in the bone even with flexible drills 149.

A most preferable method for a surgical intervention on a body part with the medical-technical measuring system is that an implant and/or a prosthesis, optionally one or several disposed drilling sleeves and the bone or bone parts to be treated are equipped with geometrical patterns and that the spatial position and orientation of a drill hole relative to the bone is measured by the measuring system.

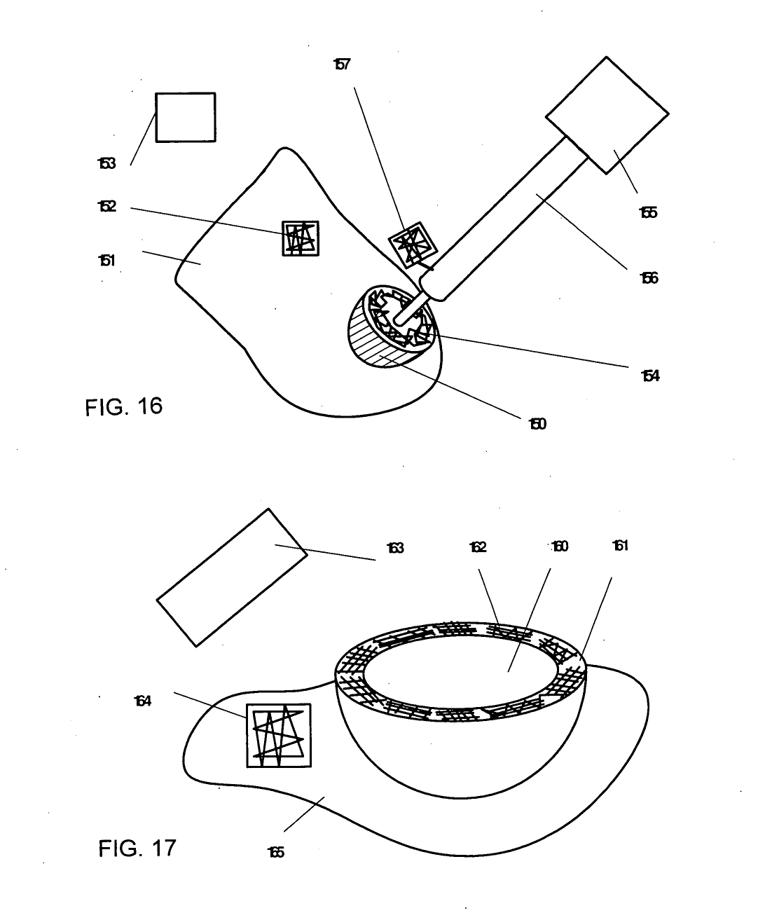

When implanting joint spocket prostheses, one must rigorously look to it that the prosthesis is set optimally into the pelvic bone material. FIG. 16 schematically shows how the position and orientation of a milling head 150 employed for this is measured by a mobile measuring system 153 relative to the registered pattern 152 anchored in the pelvic bone (pelvis) 151. The geometry of the milling head 150 and the pattern 154 relative to each other is known, for instance defined according to manufacture. The milling head 150 with the pattern 154 is clamped into the shaft 156 of the drilling apparatus 155. The measuring system 153 simultaneously measures both markers 152 and 154. The position and orientation of the milling head 150 relative to the pattern 152 on the pelvis is thus known at all times. This measurement can be carried out for instance after each milling work step (with little material excavation), until the cutout lies optimally in the bone material.

In a preferred variant, the milling head 150 does not have a pattern. For this, the pattern 157 that is mounted fix for instance on the shaft 156 of the drilling apparatus 155 is measured simultaneously with the registered pattern 152 on the pelvis 151 by the measuring system 153. The position of the milling head 150 is thereby previously calibrated with respect to the pattern 157.

A device and a method are presented in the following, in which the position and orientation of implanted joint socket prostheses 160 for instance are determined. As shown schematically in FIG. 17, the edge 161 of the socket is equipped with an adequate pattern 162 that is recognized and analyzed by the preferably mobile measuring system 163. Another pattern 164 as reference is attached for instance to the pelvic bone (pelvis 165). The position and orientation of the joint socket prostheses relative to the reference pattern on the pelvis is thus measurable. Other pattern parts 162 contain an identification and/or information regarding the geometry of the prosthesis such as diameter and center of the ball socket.

The surface topology of the prosthesis relative to the reference pattern can be measured optionally by means of the structured light.

A most preferable implementation for a surgical intervention on a body part with the medical-technical measuring system is that the laminar patterns are attached directly at an appropriate place on an implant for determining the spatial position and orientation and/or for identification and/or for geometric characterization.

The method described in the U.S. Pat. No. 6,424,856B1, in which CAS can be used for soft tissues, is substantially improved with regard to practical realization by the measures described in the following.

The measures are first of all a sufficient cover of the surface of the soft tissue with an adequate fine pattern with sufficient redundancy on a flexible foil, and secondly positioning the mobile camera system relatively close to the soft tissue.

In a preferred use, the measurements are available in defined working steps in real time. The measuring system is thus used for navigation functions.

In another preferred use, the measuring system sequentially measures the topology of the surface in defined working steps.

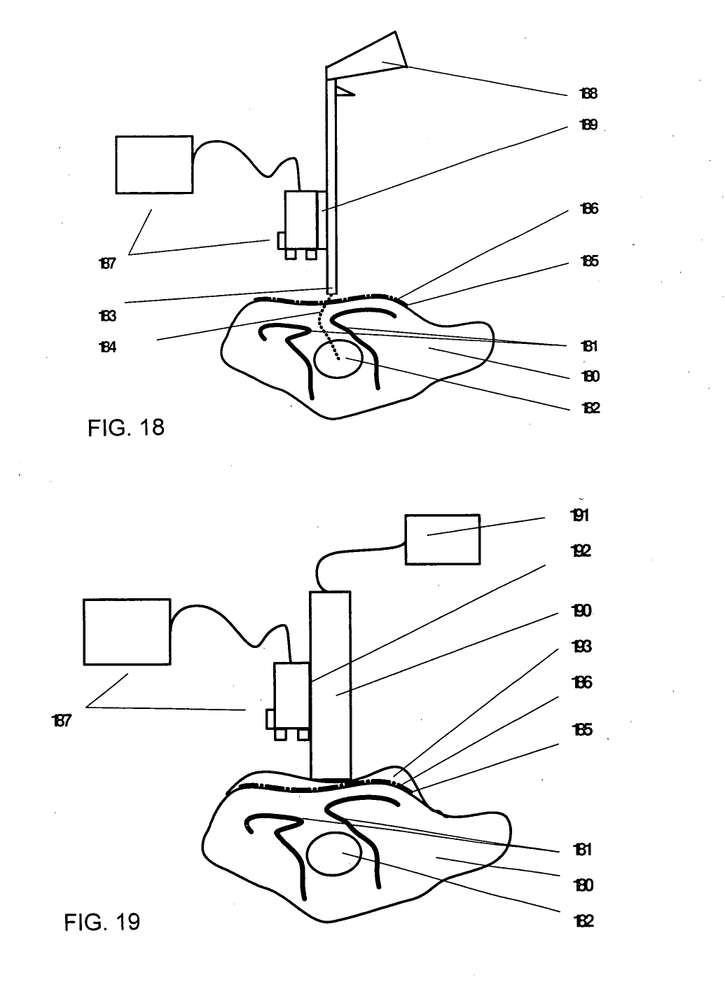

A device and a method for removing a tumor for instance from the exposed liver is shown schematically in FIG. 18. FIG. 18 represents a sectional view of the liver 180. The vessels 181 and the tumor 182 are thereby visible.

The topology of the liver is determined preoperatively by means of radiography, and if necessary, contrast agents and/or ultrasound measurements and/or MRI. The surgeon wants to lead the head 183 of the instrument through the liver tissue along the optimal path 184 toward the tumor 182 in such a manner that important vessels 181 in the organ for instance are not injured. In the prior art, the surgeon uses ultrasound technology on the exposed liver during the intervention, which often cannot be employed ergonomically in order to lead the head of the instrument.

In the present device, expansible foils 185 with applied high-contrasted geometrical patterns 186 for instance are attached to several places of the surface of the exposed liver.

Measurements of the surface topology of deformable, flexible surfaces equipped with flexible patterns can thus be performed: a method was presented at the CARS 2008 in Barcelona, but also in the U.S. Pat. No. 6,424,856B1 by Brainlab, in which the dynamical volume properties caused by external application of a force on an organ with a tumor for instance, can be modeled. The measured geometrical changes of the flexible surface of an organ caused by punctual application of a force (caused for instance by the instrument described above) and known material properties of the organ are thereby used.

The patterns or parts of it are measured by the mobile, optical camera system 187. The position and orientation of the camera system relative to the patterns is thereby known. If the camera system 187 and the surgical instrument 188 are rigidly connected to each other, by means of the adapter 189, the position and orientation of the instrument relative to the pattern 186 is also known.

After carrying out the registration, as shown below, the positions and orientations of the patterns relative to the topology of the liver are sufficiently known. At the beginning, the surgeon can thus run the instrument into the liver in a targeted manner and guide it along the optimal path 184 past the blood vessels and other important tissue parts to the desired area of the tumor. The actual surgical treatment then occurs, for instance with RF-ablation of the tumor. After the surgical intervention, the foils with the pattern are removed. Bioresorbable foils alternatively used do not have to be removed.

FIG. 19 exemplarily shows how the pattern attached to the surface of the liver is registered relative to the liver. The mobile camera system 187 is rigidly attached to the measuring head 190 of the ultrasound apparatus 191 by means of an adequate fixture 192. The measurement data of the ultrasound apparatus as well as from the optical camera system is collected and is matched by means of adequate software. The position of the patterns relative to the topology of the liver volume is thus known.

This method is completed for instance by the fact that the foil 185 with the patterns contains additional defined areas that result in a high-contrasted image with ultrasound measurements, if necessary with the use of a coupling agent 193.

Another method for registration of the pattern relative to the liver topology provides that the foils with the high-contrasted patterns also contain X-ray opaque areas. The X-ray opaque areas and thus the high-contrasted patterns on the foil are thus registered relative to the volume of the liver by means of radiography.

Surgical, navigated interventions with the technology described above are also adapted for non rigid objects such as organs, muscles and/or ligaments. With another preferred use of the described measuring system, measurements are performed in the operation field with structured light for determining the individual surface topology of several objects, which are optionally equipped with rigid and/or flexible patterns.

A most preferable method for a surgical intervention on a body part with the medical-technical measuring system is that a) the volume of the body part is captured three-dimensionally by means of an imaging method such as MRI, ultrasound or radiography, b) the position of body parts, such as blood vessels, organs, glands and tumors is described three-dimensionally, c) the work steps of the surgical intervention are defined, d) the body part is exposed in a first work step during the surgical intervention, e) rigid structures or flexible foils with geometrical patterns that are recognizable by the camera system, and secondary patterns, the position of which relative to the geometrical ones is known and which are recognizable for ultrasound measurements, CT and/or C-arm, are attached to the surface of the exposed body part, f) the geometrical pattern is registered by means of measurements with ultrasound, CT and/or C-arm with respect to the secondary pattern on the surface of the body part and/or the volume of the body part by using the previously established model, g) the optimal path of a surgical instrument is determined based on the captured volume topology and h) a surgical instrument equipped with patterns is inserted past other body parts, such as arteries and veins, on an optimal path toward the area to be treated, while being guided by the measuring system with respect to the geometrical patterns on the foil and is used there.

A most preferable method for a surgical intervention on a body part with the medical-technical measuring system is, that the surgical instrument is used for high-frequency ablation (RF-ablation).

A most preferable method for a surgical intervention on a body part with the medical-technical measuring system is, that the intervention is carried out on a liver.

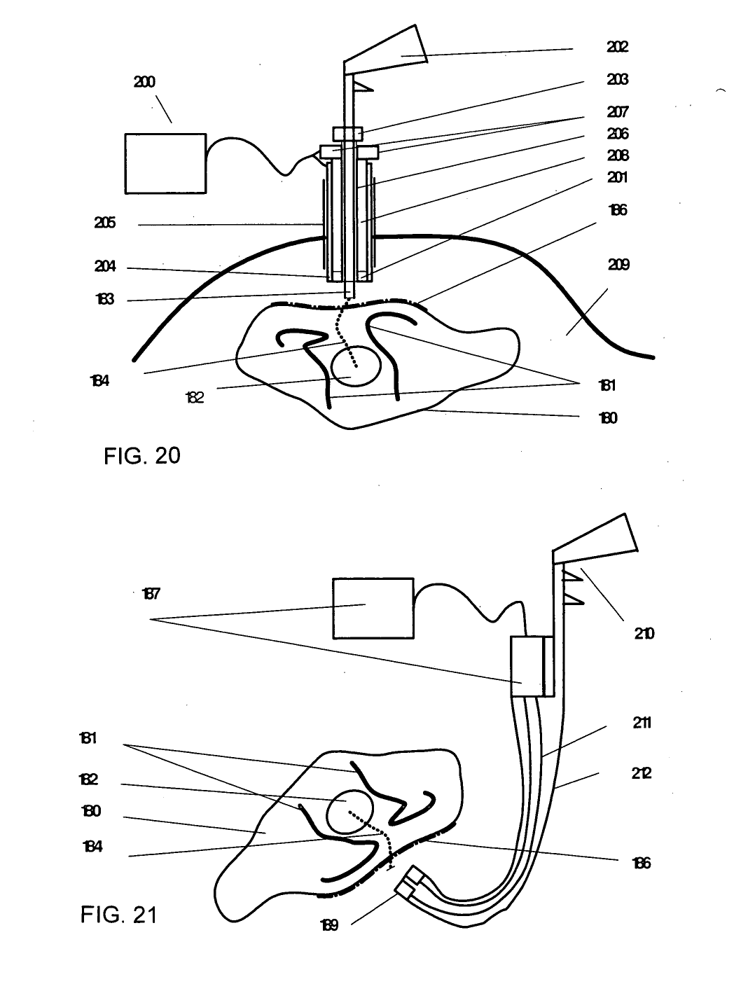

FIG. 20 exemplarily shows the device in laparoscopic surgery. In this case also a tumor for instance must be removed from the liver.

The camera system 200 has at least two (advantageously three) endoscope-type lenses 201 that are rigidly disposed for instance in a circle around the instrument 202. The sensors 207 are connected to the lenses 201 via the image conductor 208. In another preferable implementation of the device, the lenses, the sensors and a part of the electronic system is attached to the front of the endoscope.

A most preferable implementation of the medical-technical system is that the camera system has at least two endoscopes equipped with surface sensors, the lenses of which are rigidly connected to each other.

The instrument can change its position and orientation relative to the camera system. The position and possibly the orientation of the instrument in the guiding tube 206 relative to the lenses is registered for instance with a position sensor 203.

A sufficient illumination in the abdominal cavity 209 is achieved by means of the lighting device 204. The external tube 205 serves as a seal.

A sufficiently slim structure that is inserted into the abdominal cavity through the external tube 205 thus ensues.

In this case also, foils with patterns 186 for instance are attached to the surface of the liver. The registration occurs for instance by means of radiography, and X-ray opaque areas on the foil.

At the beginning, the surgeon can thus run the instrument into the liver in a targeted manner and then guide it along the optimal path 184 past the blood vessels and other important tissue parts to the desired area of the tumor. The actual surgical treatment of the tumor then occurs. After the intervention, the foils with the patterns are removed. Bioresorbable foils alternatively stay in the body and dissolve with the passage of time without harm to the patient.

Surgical interventions with flexible endoscopes/laparoscopes are just as adapted for the organ treatment with navigation described above. The operator thereby introduces the flexible tube into the patient in such a manner that he can preferably also treat an organ from a not directly accessible side. He attaches the surface markers to the organ in the desired place, then he determines the volume data of the organ together with the markers by means of an imaging system such as radiography, ultrasound or MRI and then leads the work instrument through the organ to the desired area. Changes in position and form of the organ can be recorded any time and are available for exact navigation.

Another preferred device with endoscope-type camera systems 187, 189, 211 with at least two cameras is exemplarily, schematically shown in FIG. 21. The front part of the endoscope-type camera system 189 is thereby guided around the organ 180 to be treated with adequate techniques 212 in a controlled manner by means of the control unit 210, so that the camera system can see the side septum or the back side of the organ. Appropriate patterns 186 are attached to the organ in an adapted area of the upper side of the organ. A registration is carried out for instance by means of radiography or by means of ultrasound. The surface equipped with the patterns can thus be registered three-dimensionally in relation to the inside of the organ with a tumor 182 and/or blood vessels 181 for instance. This device allows spatial navigation of surgical instruments equipped with appropriate markers behind organs or other not directly accessible body areas.

Another most preferable use of the medical-technical measuring system for a surgical intervention on a body part is for laparoscopy.

In the drawings:

FIG. 1 a schematic of a disposition of a drilling process by means of a drill sleeve,

FIG. 2a a schematic of a structure with a simple three-dimensional pattern without optional coating,

FIG. 2b a schematic of a structure with a simple planar pattern without optional coating,

FIG. 2c a schematic of a structure with a simple three-dimensional pattern with a transparent coating,

FIG. 2d a schematic of a structure with a three-dimensional structure in the shape of a blunt pyramid,

FIG. 3 a schematic of a structure with optically high-contrasted geometrical patterns on very flexible, expansible strips on a deformable object,

FIG. 4 a schematic of another structure with optically high-contrasted geometrical patterns on a very flexible, expansible foil on a deformable object,

FIG. 5 a schematic of the essential components of an active structure,

FIG. 6 a schematic of a configurable active structure with a measuring system,

FIG. 7 a schematic of a measuring system with several cameras and with an integrated light source for projecting a structured light pattern onto objects equipped with structures,

FIG. 8 a schematic of a disposition of a measuring system with three cameras equipped with periscopes,

FIG. 9 a schematic of details of a camera with a periscope,

FIG. 10 a schematic of a disposition of an optical measuring system with sterilizable lenses, sensors and electronic elements,

FIG. 11 a schematic of details of a camera with sterilizable lenses, sensors and electronic elements

FIG. 12 a schematic of a disposition of a camera system with an integrated video camera,

FIG. 13 a schematic of a disposition of a camera system with an integrated emitter and sensor for contactless diagnosis of the object,

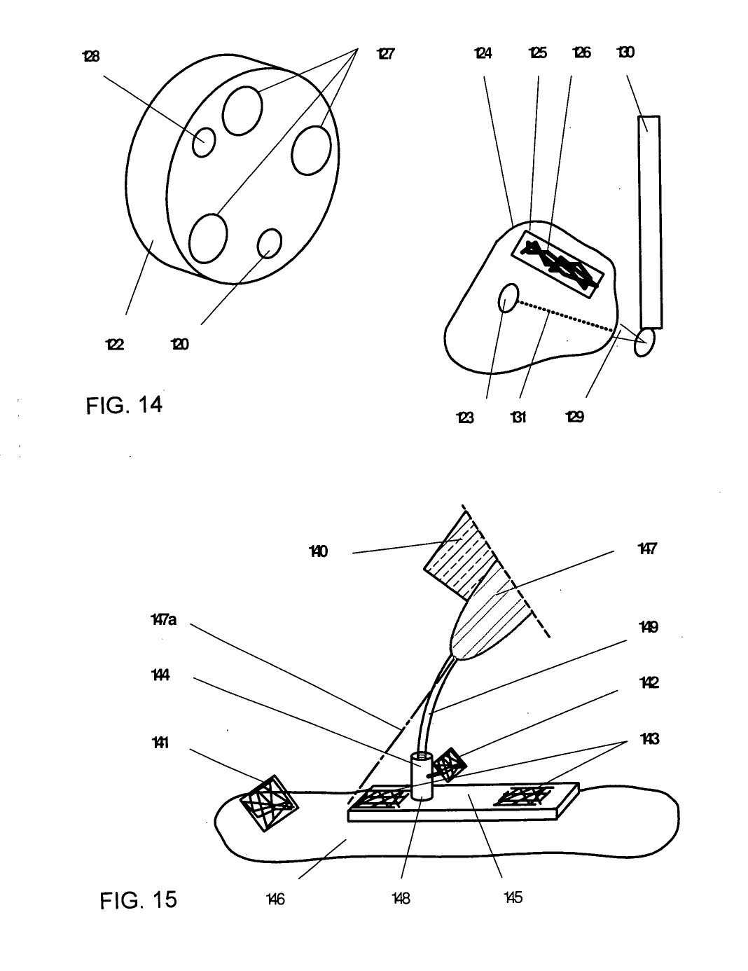

FIG. 14 a schematic of a disposition of a camera system with an emitter behind the body and a sensor integrated to the camera system for diagnosis

FIG. 15 a schematic of a mobile camera system attached to a drilling apparatus, which measures the relative orientations and positions of sleeves, plate implants, and bones equipped with patterns,

FIG. 16 a schematic of a milling head with a pattern, the place and position of which relative to the reference pattern on the pelvis is measured by the measuring system,

FIG. 17 a schematic of an implanted joint socket with a pattern, the place and position of which relative to the reference pattern on the pelvis is measured by the measuring system,

FIG. 18 a schematic of a device and a method for the removal of a tumor for instance from an exposed liver,

FIG. 19 a schematic of a registration of the pattern attached to the surface of the liver relative to the liver by means of ultrasound technology,

FIG. 20 a schematic of a device for laparoscopic surgery,

FIG. 21 a schematic of a device for laparoscopic surgery with flexible endoscopes.

1. A medical-technical measuring system with an optical camera system with at least two laminar imaging sensors, a light source rigidly connected to the camera system for projection of structured light onto objects and a structure with a laminar pattern that is attached to an object.

2. The medical-technical measuring system according to claim 1, wherein the measuring system contains lighting means for pulsed fill lighting of the object.

3. The medical-technical measuring system according to claim 1, wherein it has at least one other light source for projection of structured light onto objects.

4. The medical-technical measuring system according to claim 3, wherein the other light source has a different color than the first one.

5. The medical-technical measuring system according to claim 3, wherein the other light source projects a different structure than the first one.

6. The medical-technical measuring system according to claim 1, wherein a light beam is projected onto controlled movable mirrors rigidly connected to the camera system for projection of structured light onto objects.

7. The medical-technical measuring system according to claim 1, wherein laser light is projected onto diffractive optical elements (DOE) for projection of structured light onto objects.

8. The medical-technical measuring system according to claim 1, wherein it has a contactless radiation measuring device rigidly connected to the camera system for the ultraviolet, visible, near, mid and far infrared and/or terahertz region of the spectrum.

9. The medical-technical measuring system according to claim 8, wherein the radiation measuring device has at least one time and/or spatial resolving sensor.

10. The medical-technical measuring system according to claim 1, wherein it has at least one contactless radiation device rigidly connected to the camera system for the ultraviolet, visible, near, mid and far infrared and/or terahertz region of the spectrum.

11. The medical-technical measuring system according to claim 1, wherein it has at least one video camera rigidly connected to the camera system.

12. The medical-technical measuring system according to claim 1, wherein it has at least one display with control elements rigidly connected to the camera system.

13. The medical-technical measuring system according to claim 1, wherein respectively one periscope is disposed on the camera system between the sensor and the lens.

14. The medical-technical measuring system according to claim 1, wherein the camera system has sensors disposed or spaced in a star shape, in front of which respectively one lens is disposed.

15. The medical-technical measuring system according to claim 1, wherein the camera system has at least two endoscopes equipped with surface sensors, the lenses of which are rigidly connected to each other.

16. The medical-technical measuring system according to claim 1, wherein it has an RFID scanner rigidly connected to the camera system.

17. The medical-technical measuring system according to claim 1, wherein a) a power source, b) a data storage unit, c) a processor, d) a display with control elements as well as e) a wireless data communications equipment are disposed with the camera system as a compact unit.

18. The medical-technical measuring system according to claim 1, wherein the structure with a laminar pattern is attached to an object.

19. The medical-technical measuring system according to claim 1, wherein the structure with a laminar pattern has geometrical patterns on rigid flat or rigid three-dimensional structures.

20. The medical-technical measuring system according to claim 1, wherein the structure with a laminar pattern has geometrical and secondary patterns that can be recognized by non optical imaging systems such as an ultrasound apparatus, MRI and/or radiographic systems.

21. The medical-technical measuring system according to claim 1, wherein the patterns are disposed on flexible, deformable structures.

22. The medical-technical measuring system according to claim 1, wherein actively radiating structures are provided.

23. The medical-technical measuring system according to claim 22, wherein the active structures have an autonomous energy supply.

24. The medical-technical measuring system according to claim 1, wherein the structures are bioresorbable.

25. The medical-technical measuring system according to claim 1, wherein the laminar patterns consist of differently colored areas.

26. The medical-technical measuring system according to claim 1, wherein the laminar patterns are attached directly to implants, prostheses, milling heads and/or rasps.

27. A method for a surgical intervention on a body part with the medical-technical measuring system according to claim 1, wherein the surface of a body part to be treated is measured locally by means of structured light and is put in spatial relation with an attached structure with a pattern.

28. The method according to claim 27, wherein the body surface measured by means of structured light is registered with regard to the other body data recorded with other imaging methods such as MRI, ultrasound or radiography.

29. The method according to claim 1, wherein a part of or the entire surface of articular cartilage is locally measured in three dimensions by means of structured light and the position and size of worn out joint parts are thus localized.

30. The method according to claim 1, wherein the rigid structures or flexible foils with geometrical patterns are anatomic structures and anatomically given patterns.

31. The method according to claim 1, wherein laterally and/or time resolved contactless diagnosis information is put in relation with the spatial topology of the object equipped with patterns.

32. The method according to claim 1, wherein an implant and/or a prosthesis, optionally one or more disposed drill sleeves, and the bone or bone part to be treated are equipped with geometrical patterns and that wherein the spatial position and orientation of a drill hole relative to the bone are measured by the measuring system.

33. The method according to claim 1, wherein the laminar patterns are attached directly to an implant in the appropriate area for determining the spatial position and orientation and/or for identification and/or geometrical characterization.

34. The method according to claim 1, wherein
the position of body parts, such as blood vessel, organs, glands and tumors is described three-dimensionally,
the work steps of the surgical intervention are defined,
the body part is exposed during a first work step of the surgical intervention,
rigid structures or flexible foils with geometrical patterns that are recognizable to the optical camera system and secondary patterns, the position of which relative to the geometrical ones is known and is recognizable by ultrasound measurements, CT and/or C-arm, are attached to the surface of the exposed body part,
the geometrical pattern is registered by means of measurements with ultrasound, CT and/or C-arm relative to the secondary pattern on the surface of the body part and/or the volume of the body part by using the previously established model,
the optimal path of a surgical instrument is determined based on the recorded volume topology and
a surgical instrument equipped with patterns is inserted past other body parts, such as arteries and veins, on an optimal path toward the area to be treated, while being guided by the measuring system relative to the geometrical patterns on the foil, and is used there.

35. The method according to claim 34, wherein the surgical instrument for high-frequency ablation (RF-ablation) is used.

36. The method according to claim 34, wherein the intervention is carried out on a liver.

37. A use of a medical-technical measuring system according to claim 1 for a laparoscopy.
